(19) United States
(12) Patent Application Publication
(10) Pub. No.: US 2004/0248125 A1
(43) Pub. Date: Dec. 9, 2004

(54) Distribution of solutions across a surface

(76) Inventors: Mark A Stremler, Franklin, TN (US); Timothy S. Fisher, West Lafayette, IN (US); Frederick R. Haselton, Nashville, TN (US); David K. Schaffer, Nashville, TX (US); Mark McQuain, Nashville, TN (US)

Correspondence Address: STITES & HARBISON PLLC, 424 CHURCH STREET, SUITE 1800, NASHVILLE, TN, 37219-2376, US

(21) Appl. No.: 10/486,734

(22) PCT Filed: Aug. 12, 2002
(86) PCT No.: PCT/US02/25415

Related U.S. Application Data
(60) Provisional application No. 60/312,008, filed on Aug. 13, 2001.

(51) Int. Cl. C12Q 1/68; G05D 9/00

(52) U.S. Cl. 435/6; 137/102

(57) ABSTRACT

A system 10 is provided for improved microarray biomolecular analysis. A microarray 36 is placed in a shallow chamber 20, and an induced motion of test fluid through the chamber is achieved by a sequential series of pulses directed to a plurality of source-sink pairs.

### TECHNICAL FIELD

The present invention is directed to a system for distribution of a fluid solution across a surface to provide efficient interaction of particles carried by the solution with a plurality of points on the surface. The invention is particularly useful for biomolecular analysis wherein a solution containing test particles is distributed across a surface having a plurality of probe materials fixed in position upon the surface. It is anticipated that the predominant application is DNA microarray hybridization analysis, which relies upon interaction of DNA ‘targets’ in solution with DNA ‘probes’ fixed on the surface of a glass slide.

### BACKGROUND ART

With the success of the Human Genome Project there comes a pressing need to enhance the transport of individual macromolecules across surfaces using fluid motion so that efficient, accurate bioassays can be developed for use in cutting-edge genomic and proteomic research. An example is the use of microarrays for identifying the DNA sequences that are present in a fluid solution.

DNA microarrays are one of the most widely used methods of biomolecular screening assays. The assay is based on selective recognition between a fixed array of known “probe” DNA and a mixture of unknown “target” DNA segments in solution. Target DNA segments interact with different probes on the array and selectively bind, or hybridize, to complementary probe DNA while rejecting hybridization with non-complementary probes. DNA hybridization is extremely discriminating. The enormous power of probe-target discrimination can be exploited by large arrays of probes that enable complex mixtures of targets to be screened for tens of thousands of probe interactions in a single experiment.

DNA microarrays are typically configured in a high-density array of unique probes (thousands per cm²). The arrays are typically printed using contact deposition or ink-jet deposition techniques using liquid solutions containing unique probe DNA. Typical probe spots range from 100 microns to 250 microns in diameter, with spot densities ranging from 1000 to 6000 spots per cm².

The standard methodology for performing hybridization analysis involves “sandwiching” a drop of solution containing target molecules between two glass microscope slides, one or both of which have a microarray printed on their surface. The solution sits undisturbed in a humidity- and temperature-controlled environment for up to 48 hours. Target molecules interact with probe molecules by diffusing through the solution.

DNA microarrays and other massively parallel screening technologies are redefining the approach to discovery in biomedical research. Despite the broad appeal of this technology, the current methodology suffers from low sensitivity and poor repeatability.

While this technique is relatively easy to implement, many of the current limitations stem from the reliance on diffusive transport of the target molecules in solution. Diffusion mobility of target DNA is extraordinarily low, on the order of 10⁻⁶ to 10⁻⁷ cm²/sec (Eimer 1991; Lapham, Rife et al. 1997). Analytical analysis predicts that less that 0.003% of target DNA with a diffusion mobility of 10⁻⁶ cm²/sec will diffuse beyond 4 mm of its original location after one hour of diffusion. This means that a probe spot on a typical microarray queries the hybridization solution in the surrounding few millimeters, even after 12 to 14 hours. Although diffusion-driven movement is sufficient for small distances (micron scale), it is inadequate for the relatively large distances on microarrays (centimeter scale).

Limitations imposed by diffusion-driven target DNA movement result in inefficient use of target DNA because most targets in the hybridization solution do not come into contact with potentially complementary probes. It also decreases the sensitivity of analysis and increases the quantity of sample required to achieve detectable levels of target hybridization. Slow target movement also increases the time required for analysis because extremely long hybridization times (typically 12-24 hours) are required to achieve even limited exposure of the DNA targets to the probes. Diffusion can also make hybridization levels dependent on the probe location on the array. Probes situated at the edge of the array may query a smaller volume of hybridization solution than probes in the center. Reliance on diffusion movement over large distances also affects the accuracy microarray analysis. Smaller target DNA segments diffuse more rapidly, and so have a greater chance of interacting and hybridizing with a complementary probe on the array compared to larger segments of target DNA. Consequently, smaller target DNA molecules with higher mobility are likely to have hybridization levels artificially elevated relative to those of larger target DNA with lower mobility.

Current efforts to improve hybridization analysis include manipulating the solution to enhance target DNA transport. However, the use of small sample volumes common this these techniques presents unique challenge and opportunities for the design of novel enhancement technologies. The prior art also includes pulsed source-sink devices for the purpose of fluid mixing, but prior to the present invention the use of pulsed source-sink devices has not been proposed for any use analogous to that proposed by the present invention. U.S. Pat. No. 6,065,864 to Evans et al. discloses a pulsed source-sink device for the purpose of fluid mixing. The Evans et al. device is a microscale device that utilizes bubble valves for control of flow therethrough.

It has also been recognized that pulsed source-sink devices can generate a chaotic flow of particles as described for example in Jones et al. “Chaotic Advection in Pulsed Source-Sink Systems”, Phys. Fluids 31(3), March 1988, pp 469-485.

There is a continuing need in the art for improvement in systems for distributing fluids across microarrays, and analogous operations.

### DISCLOSURE OF THE INVENTION

A powerful mechanism for enhancing transport in laminar flow involves manipulating the bulk fluid in order to generate chaotic particle motions. The resulting ‘randomness’ of the motion breaks down barriers to transport, enabling particles to visit a larger percentage of the available fluid volume than if chaos did not occur. In the current context, the presence of chaos is beneficial because it results in particle trajectories that are not periodic, i.e., the particles never end up in the same spatial location twice. The present invention is directed to a system that uses the principles of chaotic transport in order to achieve the efficient distribution of particles in a fluid solution across a surface. This invention comprises a pulsed source-sink system that repeatedly extracts fluid from the volume covering the surface and subsequently injects this same fluid back into that volume, either at the point of extraction or at a different spatial location within that volume.

Using the said method to deliver target DNA to probes on the array will greatly enhance the efficiency, speed, and accuracy of microarray analysis. Efficiency will improve because a larger fraction (ideally all) of target DNA in solution would be queried by all the probes on the array. This will also increase detection sensitivity of low copy number target DNA and reduce the quantity of target sample required for analysis. Speed will be improved because target DNA will be delivered to probes by fluid motion, which is many orders of magnitude faster than DNA diffusion. Accuracy will also be improved because fluid motion will deliver target DNA to probes uniformly, with little regard for molecule size. Thus, different sizes of target DNA with different diffusion mobilities will have an equal chance of interacting and hybridizing with complementary probes on the array.

For purposes of discussion we will focus on DNA microarray analysis, but the method and design embodied here also apply to other screening technologies such as peptide arrays, protein arrays and antibody arrays.

The present invention includes both methods and apparatus for distributing fluid across a surface, and the systems of the present invention are particularly applicable for use in distributing a test fluid containing test particles across the surface of a microarray having an array of probe materials fixed in position on the microarray surface. One method for distributing fluid across a surface includes steps of:

(a) providing a shallow planar chamber having x and y dimensions, and having a z dimension perpendicular to the x and y dimensions, the z dimension being no greater than 1/10 of either of the x or y dimensions;

(b) providing at least one source-sink pair of fluid connections to the chamber, the source and sink of each pair being spaced along the x or y dimensions;

(c) providing within the chamber a probe surface having a plurality of probes defined thereon, the probes being spaced across the x and y dimensions of the chamber; and

(d) pulsing a test fluid through the chamber in a series of pulses via the at least one source-sink pair and thereby creating motion of the test fluid across the probe surface.

An apparatus of the present invention for distributing a fluid across a surface includes a test chamber having length and width dimensions at least an order of magnitude greater than a maximum depth dimension. The test chamber includes first and second fluid inlets and first and second fluid outlets. A probe surface is disposed in the test chamber and has a plurality of samples of probe materials located on the probe surface. A test fluid flow control assembly is connected to the fluid inlets and fluid outlets so that test fluid may be supplied to the chamber in a sequence of pulses directed to the first and second fluid inlets. The first and second fluid inlets are operably associated with the first and second fluid outlets, respectively, so that when fluid flows in the first fluid inlet fluid simultaneously flows out the first fluid outlet.

In another aspect of the present invention a microarray biomolecular analysis apparatus is provided which includes a chamber for receiving a microarray. The chamber includes at least two fluid inlets and at least two fluid outlets. A flow control system connected to the fluid inlets and fluid outlets of the chamber provides test fluid to the chamber in a sequential series of pulses including a first pulse in which fluid enters the first fluid inlet and simultaneously exits the first fluid outlet, and a second pulse in which the fluid enters the second fluid inlet and simultaneously exits the second fluid outlet.

In still another aspect of the invention a method of distributing fluid includes steps of:

(a) providing a working fluid volume;

(b) providing in the working fluid volume a probe surface having a plurality of probe samples of biological and/or chemical materials located on the probe surface;

(c) extracting at least a portion of the fluid from the working fluid volume;

(d) reinjecting at least part of the fluid extracted in step (c) back into the working fluid volume;

(e) repeating steps (c) and (d); and

(f) thereby distributing the fluid across the target surface.

Accordingly, it is an object of the present invention to provide improved systems for distribution of fluids and any particles contained therein across a surface.

Another object of the present invention is the provision of methods and apparatus for distributing test fluids across a microarray or other test surface for a biomolecular analysis of reactions between the test fluid and the materials located upon the microarray.

And another object of the present invention is the provision of systems for more rapidly conducting biomolecular analysis with microarrays or other test surfaces.

Still another object of the present invention is the provision of a system for more reliably providing uniform distribution of the test fluid across a test surface for biomolecular analysis.

Other and further objects features and advantages of the present invention will be readily apparent to those skilled in the art upon a reading of the following disclosure when taken in conjunction with the accompanying drawings.

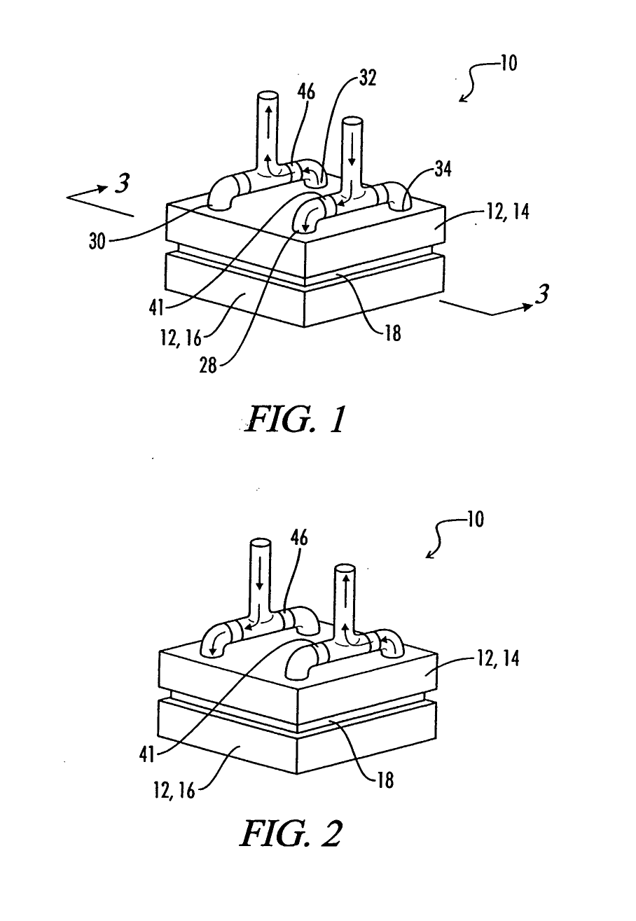

FIG. 1 is an exterior perspective view of a test system including a chamber and various conduits connected to the inlets and outlets of the chamber. The arrows indicate the direction of flow during a first pulse entering a first inlet of the chamber.

FIG. 2 is a view similar to that of FIG. 1 wherein the arrows show the direction of flow during a second pulse entering the second inlet of the chamber.

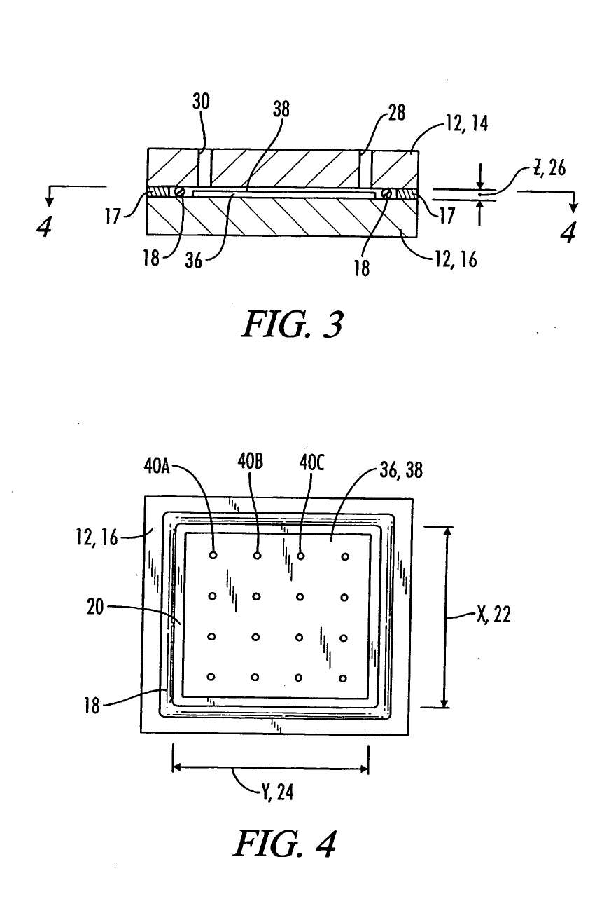

FIG. 3 is a sectioned elevation view taken along line 3-3 of FIG. 1 showing the internal construction of the test chamber and the location of a microarray therein.

FIG. 4 is a section plan view taken along line 4-4 of FIG. 3, showing the perimeter dimensions of the test chamber and of the microarray located therein.

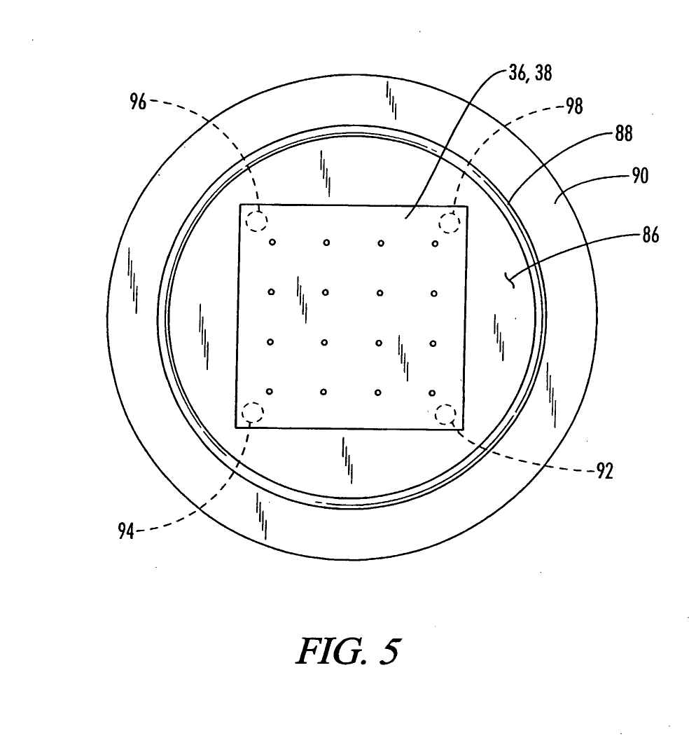

FIG. 5 is a view similar to that of FIG. 4 showing an alternative embodiment of the invention using a curvilinear or circular perimeter for the test chamber.

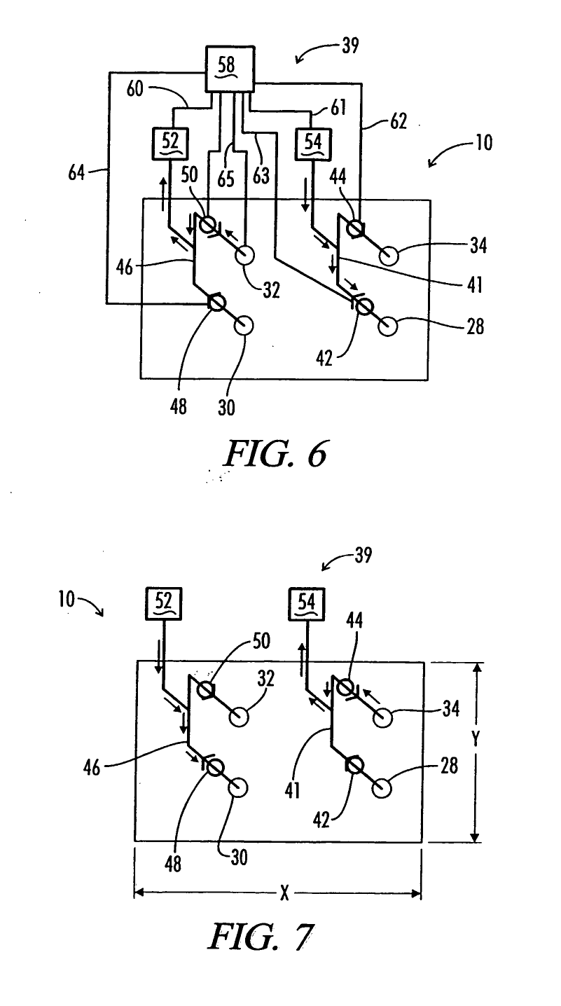

FIG. 6 is a schematic view corresponding to FIG. 1 and showing further details of the fluid flow control assembly which controls the pulsed flow of fluid to the source-sink pairs of the test chamber. The arrows depicting the direction of flow in FIG. 6 correspond to the arrows depicting the direction of flow of FIG. 1.

FIG. 7 is a view similar to that of FIG. 6, in which the arrows indicating the direction of flow correspond to the direction of flow indicated by the arrows in FIG. 2.

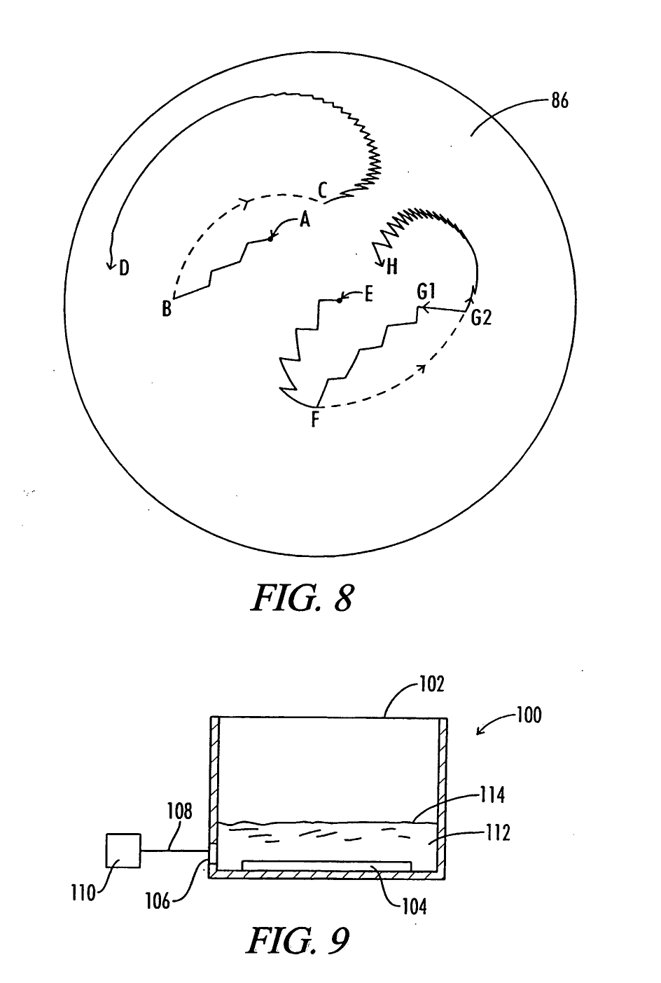

FIG. 8 is a schematic plan view of a microarray in a circular test chamber like that of FIG. 5, in which the arrows indicate an example random or chaotic path of motion of two particles carried by the test fluid relative to the fixed probe locations on the microarray.

FIG. 9 is a schematic illustration of a test chamber having an open top.

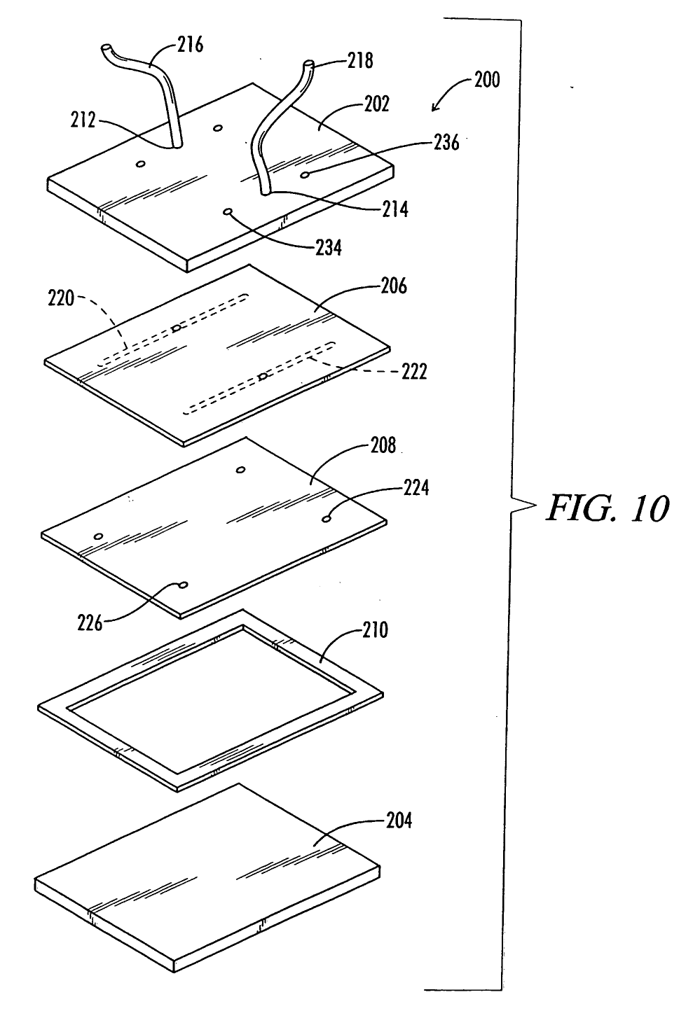

FIG. 10 is an exploded view of an alternative embodiment.

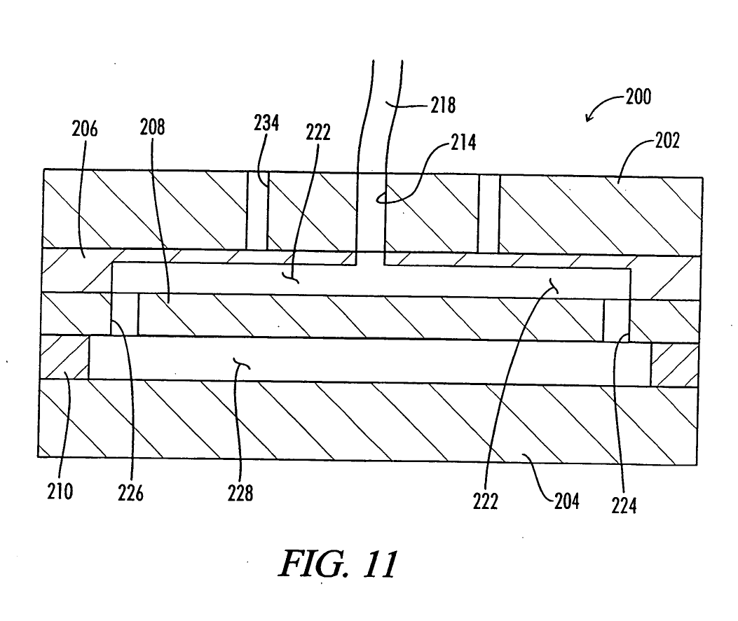

FIG. 11 is a cross sectional view of the embodiment of FIG. 10.

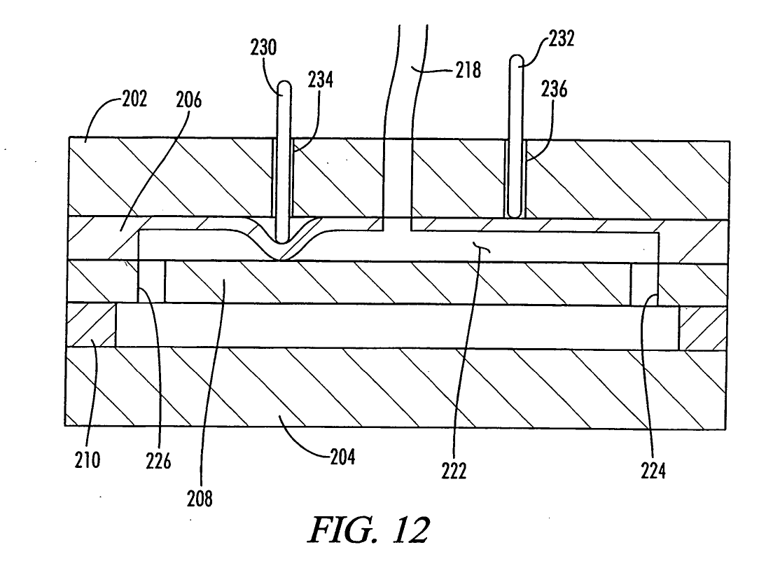

FIG. 12 is a cross sectional view like that of FIG. 11 showing actuation of the valves.

### BEST MODE FOR CARRYING OUT THE INVENTION

Referring now to the drawings, and particularly to FIGS. 1 and 2, a test system for distributing a fluid across a surface is shown and generally designated by the numeral 10. The system 10 includes a chamber housing 12 made up of a housing top plate 14 and a housing bottom plate 16. A gasket, O-ring or other sealing member 18 seals between the top and bottom plates 14 and 16 and defines a perimeter of a chamber 20 as best seen in FIG. 4.

As seen in FIG. 3, a shim 17 may be placed between the top and bottom plates 14 and 16 to control the spacing therebetween. Shim 17 is not shown in FIGS. 1 and 2.

Also, the O-ring 18 may be received in a groove (not shown) defined in either of the top and bottom plates. The top and bottom plates may be held together by screws or any other suitable fasteners (not shown).

The chamber 20 is a shallow planar chamber having x and y dimensions 22 and 24, and having a z dimension 26 perpendicular to the x and y dimensions, as best seen in FIGS. 3 and 4. The z dimension is no greater than 1/10 of either of the x or y dimensions, and more typically is no greater than 1/100 of either of the x or y dimensions.

The housing 12 has first and second inlets 28 and 30, respectively, and first and second outlets 32 and 34, respectively, defined therein and communicated with the chamber 20. The first inlet 28 may be referred to as a first source 28, and the first outlet 32 may be referred to as a first sink 32, so that the inlet and outlet pair 28 and 32 may be referred to as a first source-sink pair 28, 32. Similarly, the second inlet 30 and second outlet 34 comprise a second source-sink pair 30, 34. As is apparent in FIGS. 1-2, each source-sink pair has its respective source and sink spaced across the x and y dimensions of the chamber.

It will be understood that the x, y and z dimensions as defined herein are not intended to be arbitrarily oriented with reference to any particular geometrical feature of the chamber. They are simply used to generally represent the fact that the chamber 20 is a relatively shallow generally planar chamber having two major dimensions generally defining the planar area of the chamber and having a relatively shallow depth which is referred to as the third or z dimension. The chamber may be of any shape, two examples of which are rectangular as shown in FIG. 4 and circular as shown in FIG. 5. Any other suitable shape may be utilized. Furthermore, it will be understood that the surfaces of the chamber do not have to be flat. Various modifications such as corrugated surfaces or a curved chamber are embodied by the scope of the invention.

The chamber 20 will be sized and shaped according to the articles that are to be placed therein, such as for example a microarray like the microarray 36 best shown in FIG. 4.

Microarrays as used in biomolecular analysis are well known in lo the art. Although they may have varying dimensions, typical microarrays currently in use are manufactured from a glass slide having a length of 75 mm, a width of 25 mm, and a thickness of 1 mm, and having an array of from 100 to 25,000 microdots of biomolecular material fixed in place thereon. Other information on conventional microarray construction can be found in DNA Arrays Methods and Protocols Edited by Jang B. Rampal, Humana Press, Totowa, N.J. 2001, 264 pages, the details of which are incorporated herein by reference.

The microarray 36 has an upper surface 38 which may be referred to as a probe surface 38 having a plurality of probes such as 40A, 40B, 40C, etc. fixed or immobilized thereon. The probes 40A, 40B, 40C etc. are spaced across the x and y dimensions 22 and 24 of the chamber as schematically illustrated in FIG. 4.

After the microarray 36 is placed in the chamber 20, a test fluid is distributed across the probe surface 38 by pulsing the test fluid through the chamber 20 in a series of pulses via the source-sink pairs 28, 32 and 30, 34. This is done in a fashion, as further described below, such as to create a chaotic or random particle motion across the probe surface 38. By this approach we can make the particle motion chaotic without making the flow field itself random. Also, the particle motion need not be truly chaotic or random to achieve the benefits of the invention. In this manner the test fluid or solution is distributed across the probe surface 38 so as to provide for contact of substantially each particle of the solution with substantially each point on the test surface 38. This system distributes the solution and suspended molecules rapidly across the microarray surface 38 in a way that is largely independent of the size of the molecules carried in the test liquid fluid. The likelihood that each molecule will quickly encounter every microarray probe or test location 40A, 40B, 40C, etc. is greatly increased.

Referring now to FIGS. 6 and 7, it is seen that the system 10 includes a test fluid flow control assembly generally designated by the numeral 39. The flow control assembly is connected to the fluid inlets 28 and 30 and the fluid outlets 32 and 34 so that test fluid may be supplied to the chamber 20 in a sequence of pulses directed to the first and second inlets 28 and 30. It will be seen that the test fluid flow control assembly 39 is constructed so that the first fluid inlet 28 is operably associated with the first fluid outlet 32 so that when fluid flows in the first fluid inlet 28 fluid simultaneously flows out the first fluid outlet 32. Similarly, when fluid flows in the second fluid inlet 30 fluid simultaneously flows out the second fluid outlet 34.

The fluid flow control assembly 39 includes a first common fluid conduit 41 exterior of the chamber 20 and connecting the first fluid inlet 28 with the second fluid outlet 34. A first inlet check valve 42 is connected to the first fluid inlet 28 for preventing fluid from flowing out of the first fluid inlet 28 into the first common fluid conduit 41. An outlet check valve 44 is connected to the second fluid outlet 34 for preventing fluid from flowing from the first fluid conduit 41 into the second fluid outlet 34.

Similarly, the fluid flow control assembly 39 includes a second common fluid conduit 46 which connects second inlet 30 with first outlet 32. A second inlet check valve 48 is connected to the second inlet 30 and a second outlet check valve 50 is connected to the first fluid outlet 32.

Oscillating pumps 52 and 54 are connected to the first and second common conduits 41 and 46, respectively. The operation of pumps 52 and 54 is controlled by a controller 58 which may be a mechanical controller, an electromechanical controller, or a microprocessor controller, which is connected to pumps 52 and 54 by control cables 60 and 61 which carry control signals to the operating mechanisms of the pumps 52 and 54 in a well known manner.

The check valves 42, 44, 48 and 50 may be passive mechanical check valves such as flapper valves or ball type check valves. Alternatively they may be active solenoid type check valves in which case they will be controlled by signals communicated from controller 58 via control lines 62, 63, 64 and 65.

As schematically represented by the arrows in FIGS. 1 and 6, when displacement members (not shown) of the oscillating pumps 52 and 54 move in a first direction, (note that these pumps are moving in opposing directions) test fluid moves in the direction of the arrows so that fluid moves into inlet 28 and thus into the chamber 20, and fluid flows through the chamber 20 and out the outlet 32. During this operation, flow through second inlet 30 and second outlet 32 is prevented by the check valves 48 and 44, respectively. Then, the displacement members of operating pumps 52 and 54 reverse so that fluid flows in the direction indicated schematically by the arrows in FIGS. 2 and 7, so that a second pulse of fluid flows into second inlet 30 while fluid simultaneously flows out of second outlet 34. During this second pulse, flow through first inlet 28 and first outlet 32 are prevented by check valves 42 and 50, respectively.

Control signals from the controller 58 can vary the time interval or duration of each of the pulses, as well as the time interval between pulses in any desired manner, for example a random manner, so as to vary the flow paths of particles flowing through the test chamber 20. In general it is sufficient to use a constant time interval of each pulse and a constant time interval between each pulse to generate the necessary particle transport. It can also be appreciated that due to the construction of the test fluid flow control assembly 39, fluid that flows out of first outlet 32 can flow through the common conduit section 46 to the second inlet 30, so that at least part of the test fluid injected into the chamber 20 through the second inlet 30 is test fluid which was extracted from the chamber 20 during an earlier pulse. Similarly, due to the construction of the test fluid flow control assembly 40, fluid that flows out of second outlet 34 can flow through the common conduit section 41 to the first inlet 28, so that at least part of the test fluid injected into the chamber 20 through the second inlet 28 is test fluid which was extracted from the chamber 20 during an earlier pulse.

The systems just described can deliver a large number of pulses during a relatively short time. For example, one pulse may be delivered each second, i.e. a rate of 3600 pulses/hour. For maximum fluid distribution it may be desired to have the number of pulses equal or exceed the number of probe spots on the probe surface of the microarray. Thus for microarrays having from 100 to 25,000 probes, test times could run from a few minutes to approximately seven hours or greater.

In general the system 10 can be described as one which uses time-dependent laminar flow to efficiently distribute a given volume of a solution, and any molecules or particles suspended in this solution, across a probe surface in a high-aspect-ratio fluid chamber with a large probe surface area (along axes x and y) and a small lateral dimension (along axis z). Under proper choice of operating parameters, the flow pattern produced in the chamber 20 may be described as chaotic advection, such as described in Jones et al. “Chaotic Advection in Pulsed Source-Sink Systems”, Phys. Fluids 31(3), March 1988, pp 469-485, the details of which are incorporated herein by reference. Chaotic advection results in rapid separation of initially adjacent molecules in the test fluid, which leads to efficient distribution of the test fluid across the test surface 38 located in the chamber 20. Such flow is schematically illustrated in FIG. 8. However, it can be appreciated that chaotic motion is not necessary for the invention to enhance transport relative to diffusion in a static flow.

The primary means of achieving the desired chaotic motion is the pulsing of the fluid through the test chamber 20 by a series of source-sink pairs such as 28, 32 and 30, 34. Each source such as 28 and 30 comprises a small hole in the chamber wall through which fluid is injected, and each sink such as 32 and 34 comprises a small hole in the chamber wall through which fluid is extracted from the chamber 20. During operation of a source-sink pair such as 28 and 32, fluid is simultaneously injected into the chamber 20 through source 28 and extracted from the chamber 20 through sink 32. Fluid is moved through the chamber 20 by sequential operation of the source-sink pairs, with fluid extracted from one sink being passed to another source for reinjection. The flow patterns and particle distribution produced by such a device may be optimized by varying several aspects of the apparatus. One aspect is the variation of the location of each source and sink on any or all of the surfaces 14, 16, and 18. Another is the variation of the length of time during which each source-sink pair is operated. A third aspect is the variation of the shape and size of the chamber. A fourth aspect is the number of source-sink pairs used to pulse the flow.

One embodiment of this invention comprises a rectangular chamber 20 and two source-sink pairs as shown in FIGS. 1-4, 6 and 7. The sources and sinks are joined together in pairs by the common conduits 41 and 46, and flow is driven through the conduits 41 and 46 and the chamber 20 by two oscillating pumps 52 and 54, which may also be described as oscillating pistons 52 and 54. It is also possible for the device to operate with the elimination of one of the pumps 52 or 54. For example, flow may be driven into the inlet 28 by the oscillating pump 54 and fluid will flow out the first outlet 32 as dictated by motion of fluid through the chamber 20 and conservation of mass. Flow direction is controlled by the arrangement of check valves as previously described. Many variations on the pumps, valves and tubing can be constructed to achieve the same effect.

As noted, the chamber 20 may have a perimeter of any desired shape. For example, in FIG. 5, a circular chamber 86 is illustrated having a perimeter defined by a circular O-ring type seal 88 upon a housing base plate 90. The location of inlets which would be placed in a housing top plate (not shown) is superimposed upon the plan view of chamber 86 and the inlets are designated by numerals 92 and 94 and the outlets are designated by numerals 96 and 98. For example, in one prototype of such a circular chamber, the circular chamber 86 has a diameter of 6 inches corresponding to the x and y dimensions of the chamber, and has a thickness or depth corresponding to the z dimension of the chamber of 0.032 inches deep. For test purposes in this prototype, the sources and sinks are manually operated by inserting 0.032 inch i.d. steel tubing through self-closing rubber valves and infusing and extracting fluid using syringes. The steel tubing is then moved to alternate source-sink pairs, and fluid previously extracted from a sink is reinjected through a source.

FIG. 8 is a schematic plan view showing an illustration of two example particle trajectories generated with a numerical model of a circular domain system like that of FIG. 5. The pulse time used in FIG. 8 is fairly short. During any given pulse, the order of magnitude of a particle's motion is 1/10 of the diameter of the device. Longer pulse times move the fluid around more but require more sample volume. Also, longer pulse times are harder to illustrate clearly because particles are drawn into the sinks much more often.

In the example of FIG. 8, the first particle starts at point A, is drawn into the sink at point B, is reinjected at the source at point C, and is transported to point D after approximately 30 total pulses. The second particle starts at point E, is drawn into the sink at point F, and is reinjected at point G. At this reinjection, the particle moves down path G1 and is drawn back into the source at point F. After being reinjected at point G for the second time, the particle is transported along path G2 and moves to point H after approximately 30 total pulses.

Although the embodiments illustrated herein contain only two source-sink pairs, similar results can be produced using additional source-sink pairs.

It is also contemplated that in the broadest aspects of the invention, a single source-sink pair may be utilized to produce an improved fluid flow distribution, which may fall somewhat short of the chaotic or random particle motion which is preferred.

Furthermore, the chaotic or randomized particle motion may be influenced by more complex chamber designs which may allow for rotating the chamber relative to the test surface, and/or may allow for variation of the shape of the chamber perimeter relative to the test surface.

For example, as schematically illustrated in FIG. 9, a test chamber 100 can be designed having an open top 102 so that the volume of test solution can vary during the test. A microarray probe 104 is shown in place within the test chamber 100. The test chamber 100 can function with a single inlet/outlet 106 connected by conduit 108 to pump 110. The test solution 112 contained in the chamber 100 has an unbounded upper surface 114 which may rise and fall within the chamber 100 as fluid is injected and subsequently withdrawn from the chamber 100 by means of pump 110.

When utilizing a system like that of FIG. 9 it is desirable that the environment surrounding the system be such that evaporation of the test sample is not a problem.

A test chamber having variable volume could also be constructed using a balloon type chamber (not shown).

In the primary application of the system 10 for biomolecular analysis using microarrays, the probe molecules are immobilized on the microarray surface, and test molecules in solution are distributed across the surface. The objective of the apparatus 10 is to bring each and every suspended molecule in the test solution into close proximity with a complementary immobilized probe to allow for every possible identification event to occur in a timely manner. It will be understood, however, that while the goal of the invention is to allow contact of every suspended particle with a complementary immobilized probe material, such complete randomness is not necessary in order to achieve the objective of the invention which is the improved efficiency of distribution of such test materials across the test surface.

The Embodiment of FIGS. 10-12

Referring now to FIGS. 10-12 an alternative embodiment of the fluid distribution system is shown and generally designated by the numeral 200. The system 200 includes a housing top plate 202 and housing bottom plate 204. An elastomeric valve plate 206, an intermediate plate 208, and a gasket 210 are sandwiched between top and bottom plates 202 and 204. The assembly 200 of FIG. 10 is held together by bolts, screws, clamps or other suitable fasteners which are not shown.

FIG. 11 shows a schematic cross sectional view of the system 200.

Top plate 200 has first and second main fluid ports 212 and 214 which are connected to conduits 216 and 218.

The ports 212 and 214 are communicated with lateral passages 220 and 222 defined in the elastomeric member 206. The lateral ends of the passages 220 and 222 communicate through ports such as 224 and 226 in intermediate plate 208 with the chamber 228 which is surrounded by gasket 210.

As best seen in FIG. 12, vertical actuating rods such as 230 and 232 extend through actuating ports such as 234 and 236 so as to close either end of the passage 222 thus effectively closing ports such as 224 and 226. Thus the actuating rods 230 and 232 provide a substitute for the check valves described in the embodiment of FIGS. 1-4.

With the embodiment of FIGS. 10-12, the volume of fluid required to fill the test chamber 228 and the accompanying conduits is significantly reduced.

Thus it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. A method for distributing a fluid across a surface, comprising:
(a) providing a shallow planar chamber having x and y dimensions, and having a z dimension perpendicular to the x and y dimensions, the z dimension being no greater than 1/10 of either of the x or y dimensions;
(b) providing at least one source-sink pair of fluid connections to the chamber, the source and sink of each pair being spaced along the x and/or y dimensions;
(c) providing within the chamber a probe surface having a plurality of probes defined thereon, the probes being spaced across the x and/or y dimensions of the chamber; and
(d) pulsing a test fluid through the chamber in a series of pulses via the at least one source-sink pair and thereby creating a motion of the test fluid across the probe surface.

2. The method of claim 1, wherein:
step (b) comprises providing at least a second source-sink pair of fluid connections to the chamber.

3. The method of claim 2, wherein step (d) further comprises:
(d)(1) injecting test fluid into the chamber through the first source, for a first time interval, and simultaneously extracting test fluid from the first sink; and
(d)(2) after (d)(1), injecting test fluid into the chamber through the second source, for a second time interval, and simultaneously extracting test fluid from the second sink.

4. The method of claim 3, further comprising repeating steps (d)(1) and (d)(2).

5. The method of claim 4, further comprising varying the first and second time intervals.

6. The method of claim 3, wherein:
in step (d)(2), at least part of the test fluid injected into the chamber is test fluid which was extracted from the chamber in step (d)(1).

7. The method of claim 1, wherein the motion of test fluid across the probe surface is laminar flow.

8. The method of claim 1, further comprising:
during step (d), varying at least one boundary of the chamber.

9. The method of claim 1, wherein:
in step (c), the probe surface is a surface of a microarray having an array of biological and/or chemical probe materials immobilized on the microarray surface.

10. The method of claim 9, wherein:
in step (d), the test fluid includes a liquid solution carrying a plurality of particles of test material, and the motion of the test fluid causes a majority of the probes to be contacted by a majority of the particles of test material.

11. A system for distributing a fluid across a surface, comprising:
a test chamber having length and width dimensions at least an order of magnitude greater than a maximum depth dimension;
first and second fluid inlets to the chamber and first and second fluid outlets from the chamber;
a probe surface disposed in the chamber and having a plurality of samples of probe materials located on the probe surface; and
a test fluid flow control assembly connected to the fluid inlets and fluid outlets, so that test fluid may be supplied to the chamber in a sequence of pulses directed to the first and second fluid inlets, the first and second fluid inlets being operably associated with the first and second fluid outlets, respectively, so that when fluid flows in the first fluid inlet fluid simultaneously flows out the first fluid outlet.

12. The system of claim 11, wherein the test fluid flow control assembly further comprises:
a common fluid conduit external of the chamber and connecting the first fluid inlet with the second fluid outlet;
an inlet check valve connected to the first fluid inlet for preventing fluid from flowing out of the first fluid inlet into the common fluid conduit; and
an outlet check valve connected to the second fluid outlet for preventing fluid from flowing from the common fluid conduit into the second fluid outlet.

13. The system of claim 12, further comprising:
a second common fluid conduit external of the chamber and connecting the second fluid inlet with the first fluid outlet;
a second inlet check valve connected to the second fluid inlet; and
a second outlet check valve connected to the first fluid outlet.

14. The system of claim 13, further comprising:
at least one pump connected to the first and second common fluid conduits for sequentially injecting fluid into the first and second inlets.

15. A microarray biomolecular analysis apparatus, comprising:
a chamber for receiving a microarray, the chamber including at least two fluid inlets and at least two fluid outlets; and
a flow control system connected to the fluid inlets and fluid outlets of the chamber for providing test fluid to the chamber in a sequential series of pulses including a first pulse in which fluid enters the first fluid inlet and simultaneously exits the first fluid outlet, and a second pulse in which fluid enters the second fluid inlet and simultaneously exits the second fluid outlet.

16. The apparatus of claim 15, wherein the flow control system further comprises:
check valves associated with each of the fluid inlets and fluid outlets.

17. The apparatus of claim 15, wherein the flow control system further comprises:
at least one pump for alternatingly injecting fluid into the first and second fluid inlets.

18. The apparatus of claim 15, wherein the flow control system further comprises a pulse interval adjustment for varying a length of time during which fluid is injected during each sequential pulse.

19. A method of distributing fluid, comprising:
(a) providing a working fluid volume;
(b) providing in the working fluid volume a probe surface having a plurality of probe samples of biological and/or chemical materials located on the probe surface;
(c) extracting at least a portion of the fluid from the working fluid volume;
(d) reinjecting at least part of the fluid extracted in step (c) back into the working fluid volume;
(e) repeating steps (c) and (d); and
(f) thereby distributing the fluid across the probe surface.

20. The method of claim 19, wherein:
in step (c), fluid is extracted from the working fluid volume at a first point; and
in step (d), fluid is reinjected into the working fluid volume at the first point.

21. The method of claim 19, wherein:
in step (c), fluid is extracted from the working fluid volume at a first point; and
in step (d), fluid is reinjected into the working fluid volume at a second point different from the first point.

22. The method of claim 19, wherein the working fluid volume varies over time.

23. The method of claim 19, wherein the working fluid volume is constant.

24. The method of claim 23, wherein:
simultaneously with step (c), an equal amount of fluid is injected into the working fluid volume.
